(19) United States
(12) Patent Application Publication
(10) Pub. No.: US 2006/0008527 A1
(43) Pub. Date: Jan. 12, 2006

(54) Controlled phase composition technology as an improved process for protection of drugs

(76) Inventors: Yury Lagoviyer, St. Louis, MO (US); Syed Nasir Ali, Ballwin, MO (US); Gerard J. Moskowitz, Ballwin, MO (US)

Correspondence Address: BLACKWELL SANDERS PEPER MARTIN LLP, 720 OLIVE STREET, SUITE 2400, ST. LOUIS, MO, 63101, US

(21) Appl. No.: 11/177,762

(22) Filed: Jul. 8, 2005

Related U.S. Application Data
(60) Provisional application No. 60/586,846, filed on Jul. 9, 2004.
(60) Provisional application No. 60/598,533, filed on Aug. 3, 2004.

(51) Int. Cl. A61K 9/24

(52) U.S. Cl. 424/473

(57) ABSTRACT

The present invention relates to novel processes and compositions for protecting drugs, especially water soluble drugs in aqueous environments. More specifically, this process entails coating drugs with a controlled phase composition wax/lipid middle layer for controlling migration of the drug toward the composition's surface during preparation and a polymeric outer layer.

### CROSS REFERENCE TO RELATED APPLICATIONS

This application claims the benefit of U.S. provisional application Ser. No. 60/586,846, filed Jul. 9, 2004, and entitled Process For Protection Of Drugs, and U.S. provisional application Ser. No. 60/598,533, filed Aug. 3, 2004 and entitled Controlled Phase Composition Technology As An Improved Process For Protection Of Drugs, the disclosures of which are incorporated herein by reference.

### BACKGROUND OF INVENTION

1. Field of the Invention

The present invention relates to novel processes and compositions for protecting drugs, especially water soluble drugs in aqueous environments. More specifically, this process entails coating water soluble drugs with (1) a hydrophobic wax/glycerin ester middle layer characterized by a controlled phase composition (CPC) for controlling migration of the water soluble drug toward the composition's surface during preparation and/or more effectively coating uneven surfaces of the Active Pharmaceutical Ingredient (API) and (2) an interactive polymeric outer layer. The resultant compositions enable both efficient taste masking and controlled release of the water soluble drugs.

2. Background Art

Taste masking of drugs with disagreeable flavors is critical in obtaining patient compliance and the desired therapeutic effect. This problem is particularly acute for drugs that are soluble in water as they are rapidly released upon contact with the patient's saliva. Control of drug release rates enables improved drug efficacy and minimization of drug side effects. Water soluble drugs that require sustained release after ingestion can be particularly problematic as such drugs are often rapidly dissolved and assimilated, resulting in undesirable immediate increases in the drug dose.

A variety of processes and methods have been used in attempts to effectively protect water soluble drugs in aqueous environments to control release and/or mask unpleasant flavors. One common taste masking method is to coat the drug with layers of various polymeric coatings. Water based polymeric coatings such as Surelease™ (Ethylcellulose), Acrylic polymer and copolymers (Eudragit™, Acryl-EZE™), Gantrez Copolymers™, methylvinyl ether-maleic anhydride; Aquateric™, Cellulose Acetate Phthalate (FMC), and Eudragit™ that are commonly used to coat drugs do not effectively mask drug taste as the water soluble drugs typically migrate into these types of polymeric coating during the application process and subsequent time of storage. In addition, the crystal or solid form of the drug may be characterized by high concentration of surface defects such as growth steps or edges that are difficult to coat. While solvent based polymeric coatings such as Cellulose Acetate Phthalate, Cellulose Acetate Trimellitate, Hydroxypropylmethylcellulose Phthalate, Methylacrylic acid/ethyl acrylate copolymer, Hydroxypropylmethylcellulose acetate succinate and Polyvinyl acetate phthalate more effectively taste mask water soluble drugs, use of such polymers in manufacturing processes generate environmentally damaging byproducts and safety hazards that are undesirable (see for example FROM SOLVENT TO AQUEOUS COATINGS” Pondell, R., Drug Development & Industrial Pharmacy, 1984.)

Another approach to protection of water soluble Active Pharmaceutical Ingredients involves suspension of the API in a molten lipid matrix. This type of approach has been described in numerous publications (see for example Popplewell, et al. in U.S. Pat. No. 6,245,366). Unfortunately, some API particles remain on the surface of the beadlets when these suspensions are atomized to produce fine beadlets. Such API particles are not protected and rapidly dissolve when exposed to water, resulting in uncontrolled API release and the detection of objectionable tastes in the case of APIs with that characteristic.

An alternative approach to masking involves coating of the compounds with layers of hydrophobic materials such as lipids or waxes. Several examples of such processes are known. Kakiguchi et al (U.S. Patent Application 20030091648) describe drop wise addition of molten hydrophobic core materials to a fluidized hydrophilic core material in the presence of .beta.-form crystals. In this instance, it is suggested that the rate of drug release can be controlled by simply adjusting the thickness of the hydrophobic coating layer. However, the thickness of a given hydrophobic core material is often a poor predictor of drug release rates (see Wheatley, T. A. and Steuernagel, C. R. Latex Emulsions for Controlled Drug Delivery in Aqueous Polymeric Coatings for Pharmaceutical Dosage Forms 2^{nd} Ed. Marcel Dekker, Inc.). Moreover, simply coating a given drug with a hydrophobic layer does not always result in effective taste masking (Sznitowska et al; Acta Poloniae Pharmaceutica 57: 61, 2000). Finally, water soluble drugs are even more difficult to taste mask via more conventional techniques when small particles of ˜150 mcm or less are required since the specific surface area (i.e. surface area to volume ratio), and hence, the area to coat, rapidly increases with the decrease in the particle size of the API. In order to achieve an effective taste masking or drug release profile of small API-containing particles, additional coating material is required.

It is evident that existing processes for coating water soluble drugs either employ undesirable solvent-based coatings or fail to provide effective taste masking or adequate control of release rates as they fail to control migration of the water soluble drug to the surface of the dose form during preparation and/or inefficiently coat the highly irregular surface of the drug. In contrast to the currently available processes, the Controlled Phase Composition (CPC) Technology process and compositions described in this invention permit both effective taste masking as well as a means of controlling the rate of water soluble drug release to meet particular therapeutic requirements. More specifically, processes for identifying and deploying specific hydrophobic coating compositions that predictably result in oral dose forms with either slow, moderate or rapid rates of drug release while delivering effective taste masking are disclosed.

Small particles have large specific surface areas that need coverage. This normally requires the deposition of large quantities of coating to effectively manage the release of the drug, resulting in dilution of the API. The diluted API requires the use of larger quantities of the coated materials thereby increasing the quantity of the drug delivery system the patient must take. In the case of tablets, the tablet can become too large for effective delivery. The CPC Technology described herein permits effective coating of the API with reduced amounts of coating material. The efficiency of this process thus provides the drug in higher concentration dose forms that cannot be readily obtained through previously described methods.

### SUMMARY OF INVENTION

This invention is directed to an orally administered pharmaceutical composition which comprises an API-containing center core; a middle layer with controlled phase composition for controlling migration of said active pharmaceutical ingredient toward the composition's surface during the preparation of said pharmaceutical composition and/or conform better to the uneven surfaces of the API; and an interactive outer coating. The pharmaceutical composition is made by providing an API-containing center core; coating said active pharmaceutical ingredient-containing center core with a middle layer with controlled phase composition for controlling migration of said active pharmaceutical ingredient toward the composition's surface during the preparation of said pharmaceutical composition and/or conform better to the uneven surfaces of the API; and depositing an interactive outer coating around said center core and middle layer. In one embodiment of the invention the middle layer is selected to control the release of the API-containing center core. In a preferred embodiment of the invention the middle layer is selected to inhibit, and in some cases prevent, any migration so that the composition is effectively taste masked. In yet other preferred embodiments, the outer layer is selected to interact with the middle layer and exposed surface to deliver desired levels of permeability.

### BRIEF DESCRIPTION OF DRAWINGS

FIG. 1 is a cooling curve derived from a mixture of carnauba wax, beeswax, mono- and diglycerides that is used to determine the phase transition temperatures for this particular mixture of glycerin esters and waxes. This is the data used to generate the Phase Diagram.

FIG. 2 is a Phase Diagram derived from cooling curve data obtained from Sterotex, carnauba wax, and three different mixtures of Sterotex and Carnauba wax (75:25, 50:50, 25:75% Sterotex:Carnauba). Diagrams of this type are used to identify optimal glycerin esters/wax mixtures for the middle layers of the oral dose form to obtain effective taste masking or desired release rates.

FIG. 3 is a Phase Diagram with a Eutectic point derived from cooling curve data obtained from seven different mixtures of mono- and diglycerides matrix and Candelilla wax (approximately 90:10, 85:15, 82.5:17.5, 80:20, 75:25, 70:30 and 50:50% mono- and di-glycerides matrix to Candelilla wax).

FIG. 4 is a Phase Diagram derived from cooling curve data obtained from Carnauba Wax, a 95:5% Carnauba wax to Mono- and Diglycerides matrix and a 95:5% Carnauba wax to Mono- and Diglycerides matrix.

FIG. 5 is a dissolution kinetics diagram for a Dextromethorphan oral dose form with both the 82.5:17.5 mono- and di-glycerides matrix to Candelilla wax mixture middle layer (Curve A) and the 95:5% Carnauba wax to Mono- and Diglycerides matrix middle layer.

### DETAILED DESCRIPTION

As used herein, “controlled phase composition” refers to a mixture of waxes and/or lipids that have been characterized by means of Phase diagram to identify those mixtures that have a desired phase composition, (i.e. phase compositions that either define or are removed from the eutectic or peritectic point or any other characteristic point of said phase diagram or form a solid solution.)

“Interactive coatings” refer to various polymeric formulations that can both form and induce molecularly oriented or amorphous polymeric arrays when deposited on controlled phase compositions referred to in the present Invention.

This particular invention describes both a process for creating protected forms of drugs, especially water soluble drugs resulting in oral dose forms with predictable release profiles and/or efficient taste masking. Specific preferred oral dose form compositions derived from the application of this process are also disclosed. The oral dose forms described herein have an API-containing central core, a middle layer with controlled phase composition for controlling migration of the water soluble drug toward the composition's surface during preparation and/or conform better to the uneven surfaces of the API; and an interactive outer coating. This invention is suitable for creating protection for all forms of APIs, including but not limited to granules, e.g., material that has been treated to clump small particles into larger ones.

Many wax/lipid combinations are characterized by limited solid-state mutual solubility. Namely, when a mixture of molten waxes is allowed to solidify, phase separation occurs resulting in a heterogeneous solid comprising microscopic regions (grains) of various compositions. Typically, local diffusion coefficients at the phase grains interfaces are greater than the respective bulk values for the individual phase grains resulting in the increased permeability of the coating at the phase grains interfaces. Without being limited by theory, it is also possible that the mismatch of the Thermal Expansion Coefficients (TECs) of individual phase grains may cause micro-fissures to form at the boundaries between the grains. These micro-fissures could serve as water channels when a coated API is submersed. In any case, a process for exploiting the limited solid-state mutual solubility of wax/lipid combinations is described herein to obtain compositions that control migration of the water soluble drug toward the composition's surface and/or conform better to the uneven surfaces of the API and are tailored to the release and/or taste masking requirements of a given API.

The controlled phase composition middle layer can be further modified by addition of a hydrophobic polymer to form a spatially oriented continuum (Cuca et al in U.S. Pat. No. 5,494,681). The hydrophobic polymer material is present in the controlled phase composition middle layer in amounts of about 0.1% to about 50% and preferably about 2% to about 10% by weight of the of the total wax/lipid controlled phase composition middle layer. As discussed above, the hydrophobic polymer is present in amounts less than the wax/lipid core material that forms the controlled phase composition middle layer. The hydrophobic polymer material is preferably a material that has some solubility in the wax/lipid core material and is selected from a variety of natural polymers or derivatives thereof as well as synthetic polymers. Exemplary natural polymers include cellulose, cellulose acetate, cellulose phthalate, methyl cellulose, ethyl cellulose, zein, pharmaceutical glaze, shellac, chitin, chitosan, pectin, polypeptides, acid and base addition salts thereof, and mixtures thereof. Exemplary synthetic polymers include polyacrylates, polymethacrylates, polyvinyl acetate, acetate phthalate, polyanhydrides, poly(2-hydroxyethyl methacrylate), polyvinylalcohols, polydimethyl siloxone, silicone elastomers, acid and base addition salts thereof, and mixtures thereof. The term “hydrophobic polymer” as used herein refers to polymeric materials that are typically antagonistic to water, i.e., incapable of dissolving in water even though they may have regional areas in the molecule that have some hydrophilic properties.

The API-containing center core is selected from a group consisting of the compounds found in the following list:

ANALGESICS
Dihydrocodeine, Hydromorphone, Morphine, Diamorphine, Fentanyl, Alfentanil, Sufentanyl, Pentazocine, Buprenorphine, Nefopam, Dextropropoxyphene, Flupirtine, Tramadol, Oxycodone, Metamizol, Propyphenazone, Phenazone, Nifenazone, Paracetamol, Phenylbutazone, Oxyphenbutazone, Mofebutazone, Acetyl salicylic acid, Diflunisal, Flurbiprofen, Diclofenac, Ketoprofen, Meptazinol, Methadone, Pethidine, Hydrocodone, Meloxicam, Fenbufen, Mefenamic acid, Piroxicam, Tenoxicam, Azapropazone, Codeine.

ANTIALLERGICS
Pheniramine, Dimethindene, Terfenadine, Astemizole, Tritoqualine, Loratadine, Doxylamine, Mequitazine, Dexchlorpheniramine, Triprolidine, Oxatomide.

ANTIHYPERTENSIVE
Clonidine, Moxonidine, Methyldopa, Doxazosin, Prazosin, Urapidil, Terazosin, Minoxidil, Dihydralazin, Deserpidine, Acebutalol, Alprenolol, Atenolol, Metoprolol, Bupranolol, Penbutolol, Propranolol, Esmolol, Bisoprolol, Ciliprolol, Sotalol, Metipranolol, Nadolol, Oxprenolol, Nicardipine, Verapamil, Diltiazem, Felodipine, Nimodipine, Flunarizine, Quinapril, Lisinopril, Captopril, Ramipril, Fosinopril, Cilazapril, Enalapril.

ANTIBIOTICS
Democlocycline, Doxycycline, Lymecycline, Minocycline, Oxytetracycline, Tetracycline, Sulfametopyrazine, Ofloxacin, Ciproflaxacin, Aerosoxacin, Amoxycillin, Ampicillin, Becampicillin, Piperacillin, Pivampicillin, Cloxacillin, Penicillin V, Flucloxacillin, Erythromycin, Metronidazole, Clindamycin, Trimethoprim, Neomycin, Cefaclor, Cefadroxil, Cefixime, Cefpodoxime, Cefuroxine, Cephalexin, Cefradine.

ANTIHISTAMINES
Pseudoephedrine HCl, Phenylephrine HCl

BRONCHODILATOR/ANTI-ASTHMATIC
Pirbuterol, Orciprenaline, Terbutaline, Fenoterol, Clenbuterol, Salbutamol, Procaterol, Theophylline, Cholintheophyllinate, Theophylline-ethylenediamine, Ketofen.

ANTIARRHYTHMICS
Viquidil, Procainamide, Mexiletine, Tocainide, Propafenone, Ipratropium.

CENTRALLY ACTING SUBSTANCES
Amantadine, Levodopa, Biperiden, Benzotropine, Bromocriptine, Procyclidine, Moclobemide, Tranylcypromine, Tranylcypromide, Clomipramine, Maprotiline, Doxepin, Opipramol, Amitriptyline, Desipramine, Imipramine, Fluroxamin, Fluoxetin, Paroxetine, Trazodone, Viloxazine, Fluphenazine, Perphenazine, Promethazine, Thioridazine, Triflupromazine, Prothipendyl, Thiothixene, Chlorprothixene, Haloperidol, Pipamperone, Pimozide, Sulpiride, Fenethylline, Methylphenildate, Trifluoperazine, Thioridazine, Oxazepam, Lorazepam, Bromoazepam, Alprazolam, Diazepam, Clobazam, Clonazepam, Buspirone, Piracetam.

COUGH SUPPRESSANTS
Dextromethorphan, Guaifenesin.

CYTOSTATICS AND METASTASIS INHIBITORS
Melfalan, Cyclophosphamide, Trofosfamide, Chlorambucil, Lomustine, Busulfan, Prednimustine, Fluorouracil, Methotrexate, Mercaptopurine, Thioguanin, Hydroxycarbamide, Altretamine, Procarbazine.

ANTI-MIGRAINE
Lisuride, Methysergide, Dihydroergotamine, Ergotamine, Pizotifen.

GASTROINTESTINAL
Cimetidine, Famotidine, Ranitidine, Roxatidine, Pirenzipine, Omeprazole, Misoprostol, Proglumide, Cisapride, Bromopride, Metoclopramide.

ORAL ANTIDIABETICS
Tolbutamide, Glibenclamide, Glipizide, Gliquidone, Gliboruride, Tolazamide, Acarbose and the pharmaceutically active salts or esters of the above and combinations of two or more of the above or salts or esters thereof.). The above list is not meant to be exclusive.

An exemplary API used in the practice of this invention is Dextromethorphan hydrobromide, a therapeutic agent that is most effectively delivered in rapid release forms that are effectively taste masked. However, it should be recognized that this invention may also be usefully applied to the creation of oral dose forms of any compound that requires controlled release rates to mask its objectionable taste or to improve its pharmaceutical action. For example, Guaifenesin, Sodium Salicylate, Pseudoephedrine HCl, Phenylephrine HCl, morphine, hydromorphone, diltiazem, diamorphine and tramadol and pharmaceutically acceptable salts thereof are non-limiting examples of drugs that could be used as the API in the present invention. Especially preferred APIs used in this invention include Dextromethorphan HBr, Pseudoephedrine HCl, Phenylephrine HCl, Guaifenesin, Acetaminophen, Aspirin, Brompheniramine Maleate, Caffeine, Chlorpheniramine Maleate, Dimenhydrinate, Diphenhydramine, Ibuprofen, Naproxen, and pharmaceutically acceptable salts thereof. It is further recognized that other pharmaceutically acceptable excipients (i.e., emulsifiers, stabilizers, sweeteners, plasticizers or binders may be used in conjunction with pure API to form the API-containing center core. It is anticipated that the amount of material in the API-containing center core may vary between about 1 microgram to about 500 milligrams of material depending upon the dosage requirements of the specific API. In instances where the API-containing center core contains Dextromethorphan HBr, it is anticipated that either 7.5 mg (for children), 15 mg (for immediate release), or 30 mg (extended release) of material will be used in the center core. In each of these cases, Dextromethorphan HBr will comprise approximately 70% to 100% of the total material in the center core.

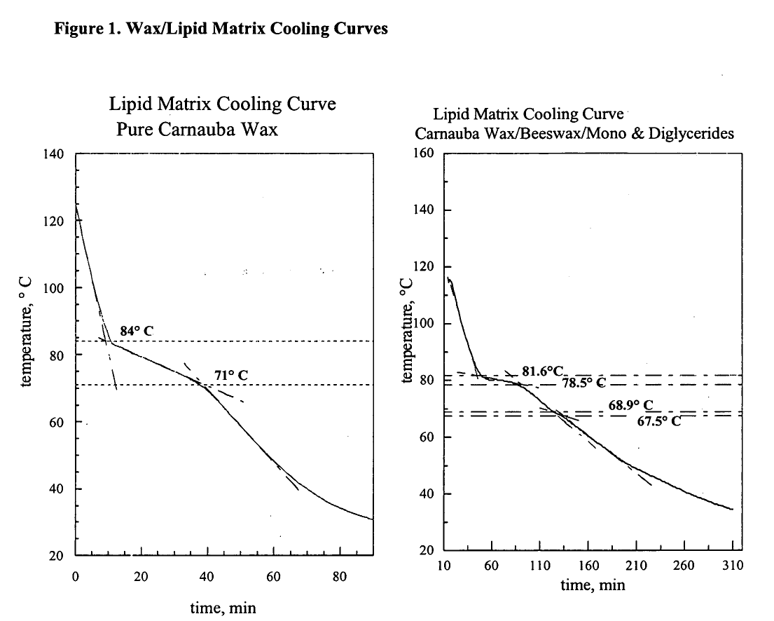

In practicing this process, the numbers of phase transitions occurring in molten mixtures of waxes and lipids which can be used as the middle layer of the composition are first determined by construction of lipid matrix cooling curves. This is accomplished by plotting the change in temperature (Y-axis) as recorded by any conventional means, such as thermal analysis/differential thermal analysis (TA/DTA) or differential scanning calorimetry (DSC) versus the change in time (X-axis) as the lipid matrix cools (FIG. 1). Phase transitions occur as the cooled wax and lipid mixture shifts from the liquid to the solid state. When a molten mixture of wax/lipid(s) is allowed to cool to ambient conditions, its temperature changes with variable rate (FIG. 1). When a phase transition (i.e., a change in the physical state of the lipid matrix from liquid to liquid plus variable composition solid, from a liquid plus variable composition solid to a liquid plus two variable composition solids, or from a liquid plus two variable composition solids occurs to a solid), the latent heat of the transition reduces the rate of temperature change of the system. On the cooling curve it is recorded as a plateau or an inflection point where each plateau or inflection point indicates a change in phase. From this data, the temperature range of each phase transition can be ascertained. If more than one transition occurs during solidification, several corresponding plateaus and/or inflection points will be seen on the graph. Each transition corresponds to the formation of a separate solid phase. The collection of cooling curve data is typically facilitated by use of TA/DTA, DSC or by simply recording the temperature of a sample in a test tube as a function of time and is well documented. A description of a cooling curve data collection experimentation is found in “Experiments in Physical Chemistry”, Shoemaker, Garland, and Nibler Sixth Ed., McGraw-Hill, 1996, pp 215-222.)

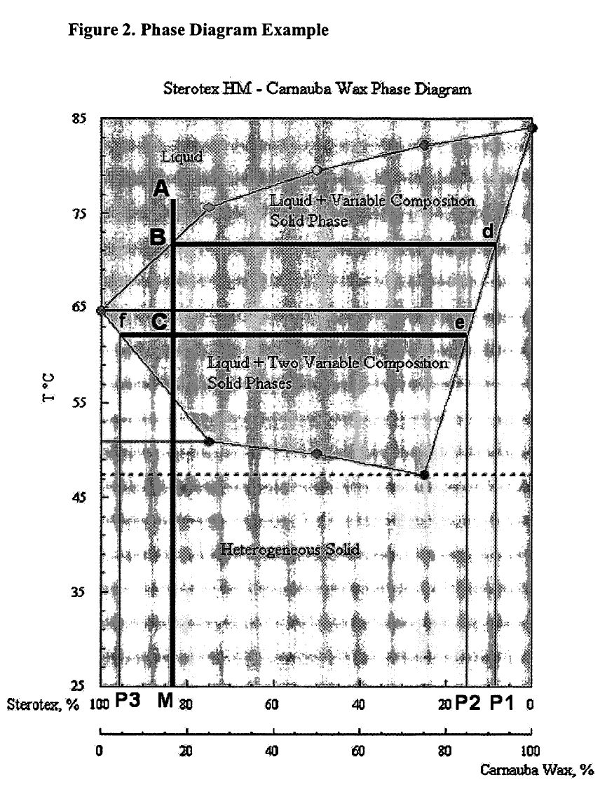

In practicing this invention, it is necessary to obtain cooling curve data for multiple mixtures of waxes and lipid where wax to lipid ratios varying from 100% (wax/lipid) to 100% (lipid/wax) to obtain a Phase Diagram (FIG. 2). As explained previously, each plateau and/or inflection point in the cooling curve for a given wax/lipid mixture represents a phase transition. Plotting temperature (Y-axis) versus the particular phase transition temperature obtained from the cooling curves for each wax/lipid mixture (X-axis) enables construction of the Phase Diagram. To obtain a useful phase transition diagram, a minimum of three distinct lipid/wax mixtures composed of for example 80 to 20, 50 to 50, and 20 to 80 percent lipids, such as glycerin esters to wax are tested in addition to the glycerin esters and the wax alone. Testing of greater than three distinct lipid to wax ratios may be pursued to obtain more refined phase transition diagrams. Lipids that are useful in the practice of this invention include fatty acids having 12 to 28 carbons, e.g., stearic acid, palmitic acid, lauric acid, eleostearic acid, etc.; fatty alcohols having from 16 to 44 carbons, (e.g., stearyl alcohol, palmitol), stearin, palmitin, lecithin, various hydrogenated vegetable oils (e.g., Sterotex HM, partially hydrogenated cottonseed oil), hydrogenated tallow, magnesium stearate and calcium and aluminum salts of palmitic and other fatty acids; various glycerin esters such as mono- and di-glycerides, partially hydrogenated soy, palm, or castor oil. Of particular and preferred use is the mono- and diglycerides preparation Dur-Em 224 (Loders Croklaan, Puchong, Malaysia). Waxes that are useful in the practice of this invention include beeswax, Candelilla wax, carnauba wax, spermaceti, paraffin wax as well as synthetic waxes e.g., those containing polyethylene, poly(ethylene glycol), poly(propylene glycol), and ethylene glycol-propylene glycol. Such a system would be predicted to form a matrix with the least amount of heterogeneity at the eutectic point, which is represented by a minimum on the solidus line (the line that separates the solid state region from the liquid state or solid/liquid state region on the phase diagram). On this particular graph that point is defined by the approximately 47 degree Centigrade transition temperature of a 75:25 mixture of Carnauba Wax to Sterotex HM. This point on the phase diagramidentifies the matrix predicted to yield the finest grain structure, hence the lowest concentration of fissures and the slowest release rate. If one desired a matrix with a faster release rate, one would choose either higher or lower Sterotex HM to Carnauba Wax ratios based on this Phase diagram.

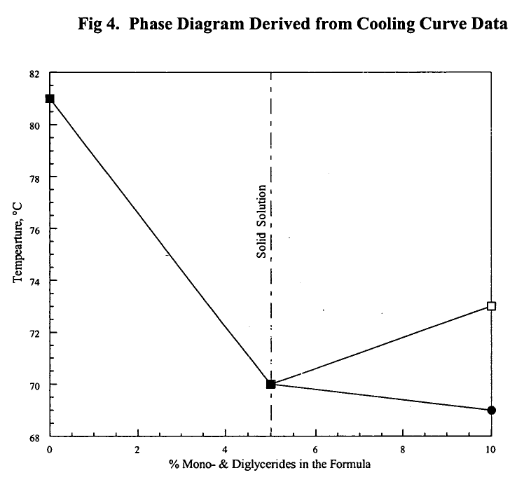

A wax/lipid system characterized by a phase diagram with a eutectic point (FIG. 4) can be chosen for a moderately to fast releasing coating. A eutectic composition would result in a matrix with a fine grain structure. The grains would be characterized by the well-matched TEC. Therefore, the concentration of fissures would be decreased (slower release rate of an API), whereas compositions located farther away from the eutectic, would result in matrices with an increased graininess. The courser grains would be characterized by the less well-matched TEC, resulting in an increased concentration of fissures (faster release rate).

Having identified the lipid to wax ratio predicted to yield the controlled phase composition middle layer that will control migration of the water soluble drug toward the composition's surface and coating the uneven surface of the API to obtain the desired release rate, the next step in the practice of this invention is to coat the API-containing center core with that specifically identified controlled phase composition middle layer of lipid and wax. The lipid and wax middle layer can be applied to the API-containing center core by any of the known means, such as encapsulation or by use of a fluid bed or coating pan apparatus. Use of a fluid bed apparatus is the best method of creating the middle layer of the oral dose forms described in this invention, is well known to those skilled in the art and is described by Mehta, A. Processing and Equipment for Aqueous Coatings in Aqueous Polymeric Coatings; pg. 387.

It is anticipated that the thickness of the wax/lipid middle layer that controls migration of the API may vary between about 0.4 mcm to about 300 mcm (micrometers) of material. In instances where the API-containing center core contains Dextromethorphan, it is anticipated that a middle layer of approximately the 10 mcm of material will be used to coat the center core. In a bulk manufacturing process, it is anticipated that between about 1% and about 90% of the dose-form composition will consist of the total wax/lipid matrix that will be deposited on the center core. In the case of the Dextromethorphan dose form, approximately 30% of the dose-form composition consists of the preferred 82.5% Candelilla wax and 17.5% mono- and diglycerides wax/lipid matrix that will be used.

The final step in the practice of this invention is to coat the controlled phase composition middle layer with an interactive outer layer. Polymeric coatings are typically preferred. Any coating that ensures that the particles of the composition maintain their integrity during further processing and/or do not release the drug until they are in either the stomach or the colon is acceptable. In the case of drugs where release in the colon is desired, the coating may be one which is pH-sensitive, redox-sensitive or sensitive to particular enzymes or bacteria, such that the coating only dissolves or finishes dissolving in the colon. Thus the oral dose form will not release the drug until it is in the colon.

The thickness of the interactive coating depends on the desired particle size of the composition and will typically be in the range 3 mcm to 50 mcm, for example between 5 mcm and 20 mcm or between 6 mcm and 15 mcm. The thickness of the particular coating used will be chosen according to the mechanism by which the coating is applied.

The interactive outer coating can both affect and be affected by the middle controlled phase composition layer. Without being limited by theory, selection of interactive coat layers may result in molecular orientation of polymers in the interactive coat and middle layer that yield dose forms with predictable and desirable permeability properties. Grains of individual solid phases in the middle controlled phase composition layer are characterized by different free surface energies. The degree of surface heterogeneity is controlled by the phase composition of the middle layer. These controlled free surface energy variations provide for controlled spatial orientation of the polymeric chains and/or their segments. In addition, the phase composition of the middle wax/lipid layer determines mismatch of thermal expansion coefficients (TEC) of the individual grains of the solid phases comprising the middle layer. This TEC mismatch determines the distribution of local stresses in the outer polymeric layer. These two phenomena (the spatially orienting action due to free surface energy distribution, and the TEC mismatch caused stress distribution) affect both permeability and the rate of swelling, disintegration, and/or dissolution of the polymeric outer coating. Recognition of these phenomena thus permits selection of an interactive outer coating with desired permeability and release characteristics.

For example, if a slower release rate of the API is desired, a eutectic or near-eutectic composition of the middle wax/lipid coating layer should be selected. Then the middle layer will be characterized be a decreased degree of heterogeneity and a reduced permeability of said layer will be attained. At the same time, the fine grain structure of the eutectic/near-eutectic composition will result in the most uniform surface energy distribution and the smallest scale of the surface features with different free surface energy values. An interactive polymeric layer such as the acrylic co-polymer Acryl-Eze® (Colorcon, West Point, Pa.) applied over a less heterogeneous controlled phase composition middle layer will be characterized by reduced stress, increased uniformity and reduced permeability.

If a faster API release rate is desired, a wax/lipid composition farther away from the eutectic point on the phase diagram should be selected. In this case, the middle layer will be characterized by a greater degree of heterogeneity. The scale of surface features will be increased, as well as the TEC mismatch between the grains of individual solid phases. This, in turn, will increase the permeability of both the middle and the interactive outer layers. In other words, an interactive coating such as the acrylic co-polymer Acryl-Eze® (Colorcon, West Point, PA) or Ethyl Cellulose (Surelease®, Colorcon, West Point, Pa.) applied over a more heterogeneous controlled phase composition middle layer will be characterized by increased stress, decreased uniformity and increased permeability. Thus, a synergistic effect between the middle wax/lipid layer with controlled phase composition and the outer interacting polymeric layer (i.e. interactive coating) is achieved.

The more uniform stress distribution of the controlled phase composition middle layer improves the deposition of that middle layer on the uneven surfaces of the API. Thus the eutectic/near eutectic composition of the middle layer provides a more uniform composition that affects both the inner core and the interactive outer polymeric layer.

A variety of classes of interactive coating materials can be effectively used in the practice of this invention. One such class is comprised of water soluble polymers such as Hydroxy Propyl Methyl Cellulose (HPMC), other cellulose derivatives, polyvinylpyrrolidone, polyvinylalcohol-polyethylene glycol graft-copolymer (Kollicoat® IR, BASF, Ludwigshafen, Germany) and amylose.

Another useful class of interactive coating materials that can be used in this invention are release-modifying water-based dispersion polymeric coatings such as Cellulose Acetate Phthalate (Aquacoat®, FMC,), Ethyl Cellulose (Surelease® Colorcon, West Point, Pa.), Acrylic copolymers such as Eudragit® dispersions (Röhm & Haas, Philadelphia, Pa.), other Acrylic copolymers such as Acryl-Eze® (Colorcon, West Point, Pa.), Polyvinyl acetate (Kollicoat® SR30D, BASF, Ludwigshafen, Germany), polyethylacrylate, methyl methacrylate (Kollicoat® EMM30D, BASF, Ludwigshafen, Germany), and methacrylic acid/ethyl acrylate copolymer Kollicoat® MAE30D (BASF, Ludwigshafen, Germany). The acrylic copolymer Acryl-Eze® is particularly useful and preferred interactive outer coating in certain embodiments of this invention where the API is Dextromethorphan and the controlled phase composition middle layer is Candelilla Wax/Mono- & Diglycerides based matrix containing 17.5% Mono- & Diglycerides (eutectic).

Other useful interactive coating materials are the pH dependent Enteric coatings that disintegrate, swell or dissolve at a pH of about 5 or above. The coatings therefore only begin to dissolve when they have left the acidic environment of the stomach and entered the small intestine. A thick layer of coating is provided which will dissolve in about 3-4 hours thereby allowing the capsule underneath to breakup only when it has reached the terminal ileum or the colon. Such a coating can be made from a variety of polymers such as cellulose acetate trimellitate, hydroxypropylmethyl cellulose phthalate, polyvinyl acetate phthalate, cellulose acetate phthalate and shellac as described by Healy in his article “Enteric Coatings and Delayed Release” Chapter 7 in Drug Delivery to the Gastrointestinal Tract, editors Hardy et al., Ellis Horwood, Chichester, 1989.

Other potentially useful enteric coatings are methylmethacrylates or copolymers of methacrylic acid and methylmethacrylate. Such materials are available as EUDRAGIT™ polymers (trademark) (Rohm Pharma, Darmstadt, Germany). Eudragits are copolymers of methacrylic acid and methylmethacrylate. Useful compositions are based on EUDRAGIT™ L100 and Eudragit S100. EUDRAGIT™ L100 dissolves at pH 6 and upwards and comprises 48.3% methacrylic acid units per g dry substance; EUDRAGIT™ S100 dissolves at pH 7 and upwards and comprises 29.2% methacrylic acid units per g dry substance. Useful coating compositions are based on EUDRAGIT™ L100 and EUDRAGIT™ S100 in the range 100 parts L100:0 parts S100 to 20 parts L100:80 parts S100. The most useful range is 70 parts L100:30 parts S100 to 80 parts L100:20 parts S100.

Yet another useful class of coatings are proteins with pH-dependent water solubility such as Casein or Zein. Such coatings typically maintain their integrity in the acidic environment of the stomach but are digested upon entry into the alkaline environment of the colon. The preferred method for coating with Casein entails solubilizing/suspending casein under alkaline conditions (i.e., in a 2N ammonium hydroxide solution with a pH greater than 9) either with or without a plasticizing agent. In contrast to previously described methods (i.e. use of a fluid bed apparatus with a solution of 10% casein and 90% APAP) that do not permit inclusion of a middle layer in a reasonably sized dosage form, this method permits coating of the API-containing center core and middle layer with an outer layer consisting of greater than 10 but less than 70% casein.

The colonic region has a high presence of microbial anaerobic organisms providing reducing conditions. Thus the coating may suitably comprise a material which is redox-sensitive. Such coatings may comprise azopolymers which can for example consist of a random copolymer of styrene and hydroxyethyl methacrylate, cross-linked with divinylazobenzene synthesized by free radical polymerization, the azopolymer being broken down enzymatically and specifically in the colon, or the polymer may be a disulphide polymer (see PCT/BE91/00006 and Van den Mooter, Int. J. Pharm. 87. 37, 1992).

Other materials which provide release in the colon are amylose, for example a coating composition can be prepared by mixing amylose-butan-lol complex (glassy amylose) with ETHOCEL™ aqueous dispersion (Milojeviic et al., Proc. Int. Symp. Contr. Rel. Bioact. Mater. 20, 288, 1993), or a coating formulation comprising an inner coating of glassy amylose and an outer coating of cellulose or acrylic polymer material (Allwood et al GB 9025373.3), calcium pectinate (Rubenstein et al., Pharm. Res., 10, 258, 1993) pectin, a polysaccharide which is totally degraded by colonic bacterial enzymes (Ashford et al.; Br Pharm. Conference, 1992, Abstract 13), chondroitin sulphate (Rubenstein et al., Pharm. Res. 9. 276, 1992), resistant starches (Allwood et al., PCT WO 89/11269, 1989), dextran hydrogels (Hovgaard and Brondsted, 3rd Eur. Symp. Control. Drug Del., Abstract Book, 1994, 87) modified guar gum such as borax modified guar gum (Rubenstein and Gliko-Kabir, S.T.P. Pharma Sciences 5, 41-46, 1995), .beta.-cyclodextrin (Sie ke et al., Eu. J. Pharm. Biopharm. 40 (suppl), 335, 1994), saccharide containing polymers, which herein includes polymeric constructs that include a synthetic oligosaccharide-containing biopolymer including methacrylic polymers covalently coupled to oligosaccharides such as cellobiose, lactulose, raffinose, and stachyose, or saccharide-containing natural polymers including modified mucopolysaccharides such as cross-linked chondroitin sulfate and metal pectin salts, for example calcium pectate (Sintov and Rubenstein PCT/US91/03014); methacrylate-galactomannan (Lehmann and Dreher, Proc. Int. Symp. Control. Rel. Bioact. Mater. 18, 331, 1991) and pH-sensitive hydrogels (Kopecek et al., J. Control. Rel. 19, 121, 1992). Resistant starches, e.g., glassy amylose, are starches that are not broken down by the enzymes in the upper gastrointestinal tract but are degraded by enzymes in the colon.

A final class of interactive coating materials that can be used in this invention are Solvent-based polymeric coatings such as methacrylic acid/ethyl acrylate copolymer Kollicoat® MAE 100P (BASF, Ludwigshafen, Germany), Cellulose Acetate Phthalate, Cellulose Acetate Trimellitate, Hydroxypropylmethylcellulose Phthalate, Methylacrylic acid/ethyl acrylate copolymer, Hydroxypropylmethylcellulose acetate succinate, Glycerol ester of maleic rosin (GMR), Pentaerythritol ester of maleic rosin (PMR) and Polyvinyl acetate phthalate.

### EXAMPLE 1

### Construction of Cooling Curve and Phase Diagram

A sample of wax/lipid matrix material was melted and placed in a glass test tube. A thermocouple was inserted in the tube. The thermocouple was connected to a temperature datalogger. The material was allowed to cool. The temperature of the sample was recorded at 1 second intervals. The plot of the sample temperature vs. time represents a cooling curve from which transition temperatures can be derived. Representative cooling curves that can be used to construct phase diagrams are shown in FIG. 1.

### EXAMPLE 2

### Formulation of a Taste Masked Oral Dose Form of Dextromethorphan

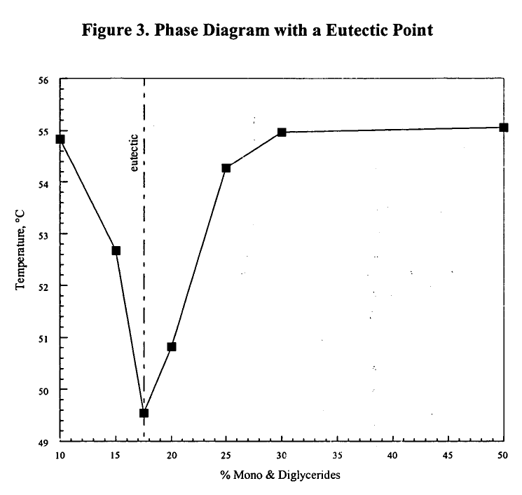

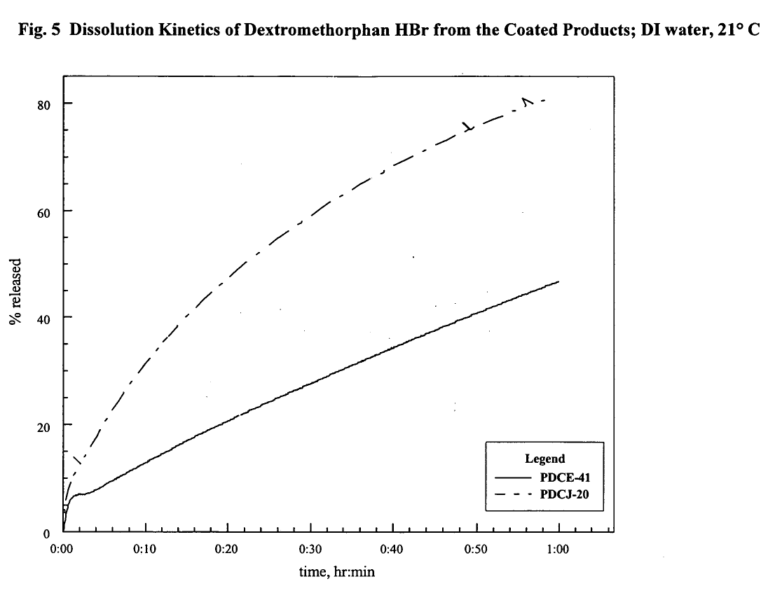

To identify a suitable lipid to wax mixture to produce a suitable controlled phase composition middle layer for the preferred taste-masked but relatively rapidly releasing Dextromethorphan oral dose form, cooling diagrams for seven distinct Candelilla Wax/Mono-& Diglycerides based matrix mixtures were first obtained. This data was in turn used to generate the Phase Diagram shown in FIG. 3. Examination of this diagram demonstrated that the Candelilla Wax/Mono- & Diglycerides matrix containing 17.5% Mono- & Diglycerides formed a eutectic and is predicted to yield a middle layer with favorable taste masking and release properties. In order to produce the taste-masked relatively rapidly releasing Dextromethorphan product, the API was coated with Candelilla Wax/Mono- & Diglycerides based matrix containing 17.5% Mono- & Diglycerides (eutectic). The matrix develops a moderate degree of heterogeneity upon cooling. The Candelilla Wax/Mono- & Diglycerides coated API is then coated with an acrylic polymer Acryl-Eze™ (Colorcon, West Point, Pa.). This product provides a coating formulation with EUDRAGIT® L100-55 (a methacrylate copolymer mixture). The product (Lot PDCJ-20) was tested for dissolution kinetics (FIG. 5) and shown to yield a desirable release rate (i.e., approximately 50% dissolution after 20 minutes in de-ionized water at 21 degrees centigrade).

### EXAMPLE 3

### Selection of Lipid and Wax Mixtures with Predicted Migration Control and Release Rate Properties

In certain instances, it may be preferable to produce oral dose forms with middle layers that promote slower release of the API. The Carnauba Wax/Mono- & Diglycerides based matrix is characterized by the Phase diagram showing a solid solution at less than 5% of Mono-& Diglycerides content (FIG. 4).

In order to produce the taste-masked relatively slow-releasing Dextromethorphan product, the API was coated with Carnauba Wax/Mono- & Diglycerides based matrix containing 5.0% Mono- & Diglycerides (solid solution). The matrix develops a low degree of heterogeneity upon cooling. The Carnauba Wax/Mono- & Diglycerides coated API is then coated with an acrylic polymer. The product (Lot PDCE-41) was tested for dissolution kinetics (FIG. 5). Release kinetics of the API (Dextromethorphan HBr) from the sample with Carnauba Wax, PDCE-41, is slower than the release kinetics of the API from the sample with Candelilla Wax, PDCJ-20. The wax/lipid coating of the Carnauba Wax derived sample PDCJ-41 is formed by solid solution and is more homogeneous than the coating of the sample PDCJ-20 (eutectic), leading to a reduced rate of API release.

In order to produce a coated product with the faster dissolution profile than the sample PDCJ-20, one would use the Candelilla/Mono- & Diglycerides based matrix. The Mono- & Diglycerides content in the matrix would be above or below eutectic point. In this case, the matrix would develop a greater degree of heterogeneity, as well as a broader distribution stresses and orientation molecular interaction fields in the outer polymeric coating leading to the faster release of an API.

### EXAMPLE 4

In order to produce a coated product with the slower dissolution profile than the sample PDCJ-20, yet faster than PDCE-41, one would use the Carnauba Wax/Mono- & Diglycerides based matrix. The Mono- & Diglycerides content in the matrix would be above the lower solid solubility level of 5%. In this case, some phase separation would develop leading to an increase in the matrix heterogeneity as compared to the solid solution state. In this case, the integrity of the matrix would be decreased leading to the faster release rate of an API.

### EXAMPLE 5

### Method for Applying a Casein Coating

To coat the dose form with casein, solid casein is dissolved under alkaline conditions (i.e., in a 2N ammonium hydroxide solution with a pH greater than 9). The outer coating can then be formed with a fluid bed apparatus using a solution of 10% casein and 90% APAP.

### EXAMPLE 6

### Taste Masked Rapidly Releasing Dextromethorphan Product

A taste-masked rapidly releasing dextromethorphan product was produced by coating the dextromethorphan with a candelilla wax/mono- and di-glycerides matrix containing 17.5% mono- and di-glycerides, corresponding to the eutectic, as illustrated in FIG. 3. The matrix composition if shown in Table 1.

TABLE 1

| Ingredient | Amount, % |
|---|---|
| Candelilla Wax (Strahl & Pitsch) | 75.5% |
| Beeswax (Strahl & Pitsch) | 5% |
| Ethoxylated Mono- and Diglycerides | 17.5% |
| (Looders Croklaan) | |
| Ethylcellulose (Dow) | 2% |

The candelilla wax and beeswax were co-melted and ethylcellulose was dissolved in the molten wax mixture at 80° C. The ethoxylated mono- and diglycerides were then dissolved in the wax mixture resulting in a molten wax matrix. The dextromethorphan HBr powder was coated with the molten Wax Matrix in a fluid bed apparatus (Glatt, GPCG-5) at 40°-45° C. to the 30% coating level. The wax coated intermediate was further coated with 30% acrylic polymer (Acryl-Eze polymer by Colorcon). The coating was performed in a GPCG-5 Fluid Bed Apparatus at 32°-34° C. product temperature. The resulting product was tested for dissolution utilizing USP Apparatus 2, equipped with rotating paddles at 50 rpm. The dissolution kinetics are illustrated in FIG. 5, PDCJ-20.

### EXAMPLE 7

### Taste Masked Slow Release Dextromethorphan Product

A taste-masked slow release dextromethorphan product was produced by coating the dextromethorphan with a candelilla wax/mono- and di-glycerides matrix containing 5.0% mono- and di-glycerides, a solid solution as illustrated in FIG. 4. The matrix composition is shown in Table 2.

TABLE 2

| Ingredient | Amount, % |
|---|---|
| Carnauba Wax (Strahl & Pitsch) | 88% |
| Beeswax (Strahl & Pitsch) | 5% |
| Mono- and Diglycerides | 5% |
| (Dur-Em 224, Looders Croklaan) | |
| Ethylcellulose (Dow) | 2% |

The carnauba wax and beeswax were co-melted to form a molten wax mixture. The ethylcellulose was then dissolved in the molten wax mixture at 80° C. The mono- and diglycerides were then dissolved in the molten wax to form a molten wax matrix.

The dextromethorphan HBr powder was coated with the molten wax matrix in a fluid bed apparatus (Glatt, GPCG-5) at 55°-60° C. until a 30% coating level is attained. The wax coated intermediate was then further coated with 30% acrylic polymer (Acryl-Eze bu Colorcon). The coating was performed in a GPCG-5 Fluid Bed Apparatus at 32°-34° C. product temperature. The resulting product was tested for dissolution utilizing USP Apparatus 2, equipped with rotating paddles at 50 rpm. The dissolution medium consisted of 900 ml deionized water at 37° C. The dissolution kinetics for the resulting product are illustrated FIG. 5, PDCE-41.

### EXAMPLE 8

### Taste Masked Slow Release Phenylephrine Product

A taste masked slow release phenylephrine product is made according to the method of Example 7, substituting phenylephrine as the API.

Having described the invention in detail, those skilled in the art will appreciate that modifications may be made of the invention without departing from its' spirit and scope. Therefore, it is not intended that the scope of the invention be limited to the specific embodiments described. Rather, it is intended that the appended claims and their equivalents determine the scope of the invention.

1. An orally administered pharmaceutical composition which comprises:
a. An active pharmaceutical ingredient-containing center core;
b. a controlled phase composition middle layer for controlling migration of said active pharmaceutical ingredient toward the composition's surface during the preparation of said pharmaceutical composition; and
c. an outer coating.

2. The composition as recited in claim 1 wherein the active pharmaceutical ingredient-containing center core comprises a water soluble compound.

3. The composition as recited in claim 1 wherein the active pharmaceutical ingredient containing center core comprises a compound selected from a group consisting of Dextromethorphan HBr, Pseudoephedrine HCl, Phenylephrine HCl, Guaifenesin, Acetaminophen, Aspirin, Brompheniramine Maleate, Caffeine, Chlorpheniramine Maleate, Dimenhydrinate, Diphenhydramine, Ibuprofen, Naproxen, and pharmaceutically acceptable salts thereof.

4. The composition as recited in claim 1 wherein the active pharmaceutical ingredient-containing center core is Dextromethorphan.

5. The composition as recited in claim 1 wherein the active pharmaceutical ingredient-containing center core comprises a compound selected from a group consisting of (a) Dextromethorphan HBr, Pseudoephedrine HCl, Phenylephrine HCl, Guaifenesin, Acetaminophen, Aspirin, Brompheniramine Maleate, Caffeine, Chlorpheniramine Maleate, Dimenhydrinate, Diphenhydramine, Ibuprofen, Naproxen, and pharmaceutically acceptable salts thereof; and (b) pharmaceutically acceptable excipients.

6. The composition as recited in claim 1 wherein the active pharmaceutical ingredient-containing center core comprises Dextromethorphan HBr and pharmaceutically acceptable excipients.

7. The composition as recited in claim 1 wherein the active pharmaceutical ingredient-containing center core comprises Dextromethorphan and excipients such as starch, Povidone, flavors, and Ethylcellulose.

8. The composition as recited in claim 1 wherein the controlled phase composition middle layer is selected from a group consisting of one or more waxes, one or more lipids, and wax/lipid mixtures, polyethylene and polypropylene synthetic waxes and esters thereof, and mixtures thereof.

9. The composition as recited in claim 8 wherein the controlled phase composition middle layer is selected from the group consisting of beeswax, candelilla wax, carnauba wax, spermaceti, paraffin wax, synthetic waxes, fatty acids having 12 to 28 carbons, fatty alcohols having from 16 to 44 carbons, mono- and diglycerides, partially hydrogenated oils of soy, cottonseed, palm, sunflower, castor and pharmaceutically acceptable salts and esters thereof.

10. The composition of claim 9 wherein the fatty acids are selected from the group consisting of stearic acid, palmitic acid, lauric acid, and eleostearic acid.

11. The composition of claim 9 wherein the fatty alcohols are selected from the group consisting of stearyl alcohol, palmitol, stearin, palmitin, lecithin, hydrogenated cottonseed soy, palm, castor, cocoa, synthetic cocoa butter, rapeseed, glycerin esters, hydrogenated tallow, and magnesium stearate.

12. The composition of claim 9 wherein the synthetic wax contains polyethylene, poly(ethylene glycol), poly(propylene glycol), and ethylene glycol-propylene glycol.

13. The composition as recited in claim 8 wherein the controlled phase composition middle layer is Candelilla wax and mono- and di-glycerides.

14. The composition as recited in claim 1 wherein the coating comprises a water suspendable, emulsifiable polymer selected from a group consisting of Cellulose Acetate phthalates, ethyl cellulose, acrylic copolymers, polyvinyl acetate polyethylacrylate, methyl methacrylate and methacrylic acid/ethyl acrylate copolymers.

15. The composition as recited in claim 1 wherein the coating comprises a polymer selected from a group consisting of B-cyclodextrins, pectin, chitosan or chitin.

16. The composition as recited in claim 1 wherein the coating is a protein selected from a group consisting of Casein or Zein.

17. The composition as recited in claim 1 wherein the coating is selected from a group consisting of cellulose acetate trimellitate, hydroxypropylmethyl cellulose phthalate, hydroxypropylmethyl cellulose acetate, hydroxypropylmethyl cellulose acetate succinate polyvinyl acetate phthalate, cellulose acetate phthalate and shellac; methylmethacrylates or copolymers of methacrylic acid and methylmethacrylate; random copolymers of styrene and hydroxyethyl methacrylate cross-linked with divinylazobenzene; disulphide polymers; amylose-butan-lol complex (glassy amylose) with ETHOCEL™ aqueous dispersion calcium mixtures; pectinate, pectin, calcium pectinate, chondroitin sulphate, resistant starches, dextran hydrogels, modified guar gum such as borax modified guar gum, beta.-cyclodextrin saccharide containing polymers, methacrylic polymers covalently coupled to oligosaccharides such as cellobiose, lactulose, raffinose, and stachyose, or saccharide-containing natural polymers including modified mucopolysaccharides such as cross-linked chondroitin sulfate and metal pectin salts, for example calcium pectate, methacrylate-galactomannan and pH-sensitive hydrogels.

18. The composition as recited in claim 1 wherein the coating is a solvent-based polymeric composition selected from a group consisting of methacrylic acid/ethyl acrylate copolymers, Cellulose Acetate Phthalate, Cellulose Acetate Trimellitate, Hydroxypropylmethylcellulose Phthalate, Methylacrylic acid/ethyl acrylate copolymer, Hydroxypropylmethylcellulose acetate succinate, Glycerol ester of maleic rosin (GMR), Pentaerythritol ester of maleic rosin (PMR) and Polyvinyl acetate phthalate.

19. The composition as recited in claim 1 wherein the coating is a water soluble polymer selected from a group consisting of Hydroxy Propyl Methyl Cellulose (HPMC), other cellulose derivatives, polyvinylpyrrolidone, polyvinylalcohol-polyethylene glycol graft-copolymer and amylose.

20. The composition as recited in claim 1 wherein the coating is Casein.

21. The composition as recited in claim 1 wherein said controlled phase composition middle layer comprises a composition which inhibits migration of said active pharmaceutical ingredient toward the composition's surface during the preparation of said pharmaceutical composition.

22. An orally administered pharmaceutical composition which comprises:
a. An active pharmaceutical ingredient-containing center core which comprises Dextromethorphan HBr;
b. a controlled phase composition middle layer comprised of 82.5% Candelilla wax and 17.5% mono- and diglycerides; and
c. an outer coating comprised of acrylic copolymers.

23. A process for preparing an orally administered pharmaceutical composition or intermediate which comprises:
i. providing an active pharmaceutical ingredient-containing center core;
ii. coating said center core with a controlled phase composition middle layer for controlling migration of said active pharmaceutical ingredient toward the composition's surface during the preparation of said pharmaceutical composition; said middle layer is selected from a group consisting of one or more waxes, one or more lipids and wax/lipid(s) mixtures; and
iii. depositing upon said center core and middle layer an outer coating.

24. A process according to claim 23 wherein said controlled phase composition middle layer inhibits migration of said active pharmaceutical ingredient toward the composition's surface during the preparation of said pharmaceutical composition.

25. A process according to claim 23 which further comprises the step of producing phase diagrams of the composition of the middle layer and choosing the specific composition of the middle layer so that migration of said active pharmaceutical ingredient toward the surface is inhibited.

26. A process according to claim 25 wherein the composition of the controlled phase composition middle layer is determined by construction of a phase diagram having three or more data points for a given wax/lipid mixture and identifying from the phase diagram the wax lipid compositions desired.

27. A process according to claim 25 wherein said controlled phase composition middle layer prevents migration of said active pharmaceutical ingredient toward the composition's surface during the preparation of said pharmaceutical composition.

28. A process according to claim 23 wherein the active pharmaceutical ingredient-containing center core comprises a water soluble compound.

29. A process according to claim 23 wherein the active pharmaceutical ingredient containing center core comprises a compound selected from a group consisting of: Dextromethorphan HBr, Pseudoephedrine HCl, Phenylephrine HCl, Guaifenesin, Acetaminophen, Aspirin, Brompheniramine Maleate, Caffeine, Chlorpheniramine Maleate, Dimenhydrinate, Diphenhydramine, Ibuprofen, Naproxen, and pharmaceutically acceptable salts thereof.

30. A process according to claim 23 wherein the active pharmaceutical ingredient-containing center core is Dextromethorphan HBr.

31. A process according to claim 23 wherein the active pharmaceutical ingredient-containing center core comprises a compound selected from a group consisting of (a) Dextromethorphan HBr, Pseudoephedrine HCl, Phenylephrine HCl, Guaifenesin, Acetaminophen, Aspirin, Brompheniramine Maleate, Caffeine, Chlorpheniramine Maleate, Dimenhydrinate, Diphenhydramine, Ibuprofen, Naproxen, and pharmaceutically acceptable salts thereof and (b) pharmaceutically acceptable excipients.

32. A process according to claim 23 wherein the active pharmaceutical ingredient-containing center core comprises Dextromethorphan HBr and pharmaceutically acceptable excipients.

33. A process according to claim 23 wherein the active pharmaceutical ingredient-containing center core comprises Dextromethorphan HBr and starch, Povidone, flavors, and Ethylcellulose.

34. A process according to claim 23 wherein the controlled phase composition middle layer is selected from a group consisting of one or more waxes, one or more lipid(s) and wax/lipid mixtures.

35. A process according to claim 34 wherein the controlled phase composition middle layer is selected from the group consisting of beeswax, candelilla wax, carnauba wax, spermaceti, paraffin wax, synthetic wax, fatty acids having 12 to 28 carbons, fatty alcohols having from 16 to 44 carbons, glycerin esters such as mono- and diglycerides and pharmaceutically acceptable salts and esters thereof.

36. A process according to claim 35 wherein the fatty acids are selected from the group consisting of stearic acid, palmitic acid, lauric acid, and eleostearic acid.

37. A process according to claim 35 wherein the fatty alcohols are selected from the group consisting of stearyl alcohol, palmitol, stearin, palmitin, lecithin, hydrogenated cottonseed oil, hydrogenated tallow, and magnesium stearate.

38. A process according to claim 35 wherein the synthetic wax is selected from the group consisting of polyethylene, poly(ethylene glycol), poly(propylene glycol), and ethylene glycol-propylene glycol.

39. A process according to claim 34 wherein the controlled phase composition middle layer is 82.5% Candelilla wax and 17.5% mono- and diglycerides.

40. A process according to claim 23 wherein the coating comprises a water soluble polymer.

41. A process according to claim 23 wherein the coating comprises a water suspendable, emulsifiable polymer selected from a group consisting of Cellulose Acetate phthalates, ethyl cellulose, acrylic copolymers, polyvinyl acetate polyethylacrylate, methyl methacrylate and methacrylic acid/ethyl acrylate copolymers.

42. A process according to claim 23 wherein the coating comprises a solvent-based polymeric composition selected from a group consisting of methacrylic acid/ethyl acrylate copolymers, Cellulose Acetate Phthalate, Cellulose Acetate Trimellitate, Hydroxypropylmethylcellulose Phthalate, Methylacrylic acid/ethyl acrylate copolymer, Hydroxypropylmethylcellulose acetate succinate, Glycerol ester of maleic rosin (GMR), Pentaerythritol ester of maleic rosin (PMR) and Polyvinyl acetate phthalate.

43. A process according to claim 23 wherein the coating comprises a polymer selected from a group consisting of B-cyclodextrins, pectin, chitosan or chitin.

44. A process according to claim 23 wherein the coating is a protein selected from a group consisting of Casein or Zein.

45. A process according to claim 23 wherein the coating is selected from a group consisting of cellulose acetate trimellitate, hydroxypropylmethyl cellulose phthalate, polyvinyl acetate phthalate, cellulose acetate phthalate and shellac; methylmethacrylates or copolymers of methacrylic acid and methylmethacrylate; random copolymers of styrene and hydroxyethyl methacrylate cross-linked with divinylazobenzene; disulphide polymers; amylose-butan-lol complex (glassy amylose) with ETHOCEL™ aqueous dispersion calcium mixtures; pectinate, pectin, calcium pectinate, chondroitin sulphate, resistant starches, dextran hydrogels, modified guar gum such as borax modified guar gum, beta.-cyclodextrin saccharide containing polymers, methacrylic polymers covalently coupled to oligosaccharides such as cellobiose, lactulose, raffinose, and stachyose, or saccharide-containing natural polymers including modified mucopolysaccharides such as cross-linked chondroitin sulfate and metal pectin salts, for example calcium pectate, methacrylate-galactomannan and pH-sensitive hydrogels.

46. A process according to claim 23 wherein the coating is an acrylic polymer or copolymer.

47. A process according to claim 23 wherein the coating is casein.

48. A process according to claim 23 wherein the orally administered form is selected from a group consisting of pills, capsules, or film tabs.

49. A process according to claim 23 wherein said composition comprises:
a. An active pharmaceutical ingredient-containing center core which comprises Dextromethorphan HBr;
b. a controlled phase composition middle layer comprised of 82.5% Candelilla wax and 17.5% mono- and diglycerides; and
c. an outer coating comprised of acrylic copolymers.

50. A process according to claim 23 wherein said outer coating is a protein.

51. A process according to claim 50 wherein said outer coating is selected from the group consisting of casein or Zein.

52. A process according to claim 50 wherein said protein is insoluble in acidic media but is soluble in basic media.

53. A process according to claim 52 which further comprises dissolving said protein in basic media having a pH above about 9.0 and applying said protein to said center core and middle layer via fluid bed.

54. A process according to claim 53 wherein said basic media is ammonium hydroxide.

55. A process for preparing an orally administered pharmaceutical composition which comprises:
i. providing an active pharmaceutical ingredient-containing center core;
ii. coating said center core with a controlled phase composition middle layer for controlling migration of said active pharmaceutical ingredient toward the composition's surface during the preparation of said pharmaceutical composition; said controlled phase composition middle layer is selected from a group consisting of one or more waxes, one or more lipids and wax/lipid mixtures;
iii. dissolving a protein which is insoluble in acid in a basic solvent having a pH greater than about 9.0; and
iv. applying said protein to said center core and middle layer via fluid bed.

56. A process according to claim 26 wherein the wax/lipid composition is identified as that having unlimited solubility in the solid state in order to obtain a slow release rate.

57. A process according to claim 26 wherein the wax/lipid composition is identified as that having a eutectic point in order to obtain slow release rate.

58. A process according to claim 26 wherein the wax/lipid composition is identified as that having the wax/lipid compositions that are not located at the eutectic point in order to obtain a faster release rate.

59. The composition of claim 8 wherein the controlled phase composition middle layer is further modified by inclusion of hydrophobic polymer material in amounts of about 0.1% to about 50% by weight of the total wax/lipid controlled phase composition middle layer to form a spatially oriented continuum

60. The composition of claim 8 wherein the controlled phase composition middle layer is further modified by inclusion of hydrophobic polymer material in amounts of about 2% to about 10% by weight of the total wax/lipid controlled phase composition middle layer to form a spatially oriented continuum.

61. The composition of claim 59, wherein the hydrophobic polymer material selected from the group consisting of natural polymers and synthetic polymers.

62. The composition of claim 59, wherein the natural polymer is selected from the group consisting of cellulose, cellulose acetate, cellulose phthalate, methyl cellulose, ethyl cellulose, zein, pharmaceutical glaze, shellac, chitin, chitosan, pectin, polypeptides, acid and base addition salts thereof, and mixtures thereof.

63. The composition of claim 59, wherein the synthetic polymer is selected from the group consisting of polyacrylates, polymethacrylates, polyvinyl acetate, polyvinyl acetate phthalate, polyanhydrides, poly(2-hydroxyethyl methacrylate), polyvinylalcohols, polydimethyl siloxone, silicone elastomers, acid and base addition salts thereof, and mixtures thereof.

64. A process according to claim 23, wherein the controlled phase composition middle layer is further modified by inclusion of hydrophobic polymer material in amounts of about 0.1% to about 50% by weight of the of the total wax/lipid controlled phase composition middle layer to form a spatially oriented continuum.

65. A process according to claim 23, wherein the controlled phase composition middle layer is further modified by inclusion of hydrophobic polymer material in amounts of about 2% to about 10% by weight of the total wax/lipid controlled phase composition middle layer to form a spatially oriented continuum

66. The process according to claim 65, wherein the hydrophobic polymer material selected from the group consisting of natural polymers and synthetic polymers.

67. The process according to claim 65, wherein the natural polymer is selected from the group consisting of cellulose, cellulose acetate, cellulose phthalate, methyl cellulose, ethyl cellulose, zein, pharmaceutical glaze, shellac, chitin, chitosan, pectin, polypeptides, acid and base addition salts thereof, and mixtures thereof

68. The process according to claim 65, wherein the synthetic polymer is selected from the group consisting of polyacrylates, polymethacrylates, polyvinyl acetate, polyvinyl acetate phthalate, polyanhydrides, poly(2-hydroxyethyl methacrylate), polyvinylalcohols, polydimethyl siloxone, silicone elastomers, acid and base addition salts thereof, and mixtures thereof.
